(19) United States
(12) Patent Application Publication
(10) Pub. No.: US 2018/0199823 A1
(43) Pub. Date: Jul. 19, 2018

(54) PHYSIOLOGICAL SIGNAL MEASUREMENT DEVICE AND BLOOD OXYGEN CONCENTRATION ALGORITHM APPLIED THEREIN

(71) Applicant: Cheng Uei Precision Industry Co., Ltd., New Taipei City (TW)

(72) Inventor: James Lee, New Taipei City (TW)

(21) Appl. No.: 15/657,685

(22) Filed: Jul. 24, 2017

(30) Foreign Application Priority Data
Jan. 17, 2017 (TW) 106101593

(51) Int. Cl. A61B 5/0205; A61B 5/1455; A61B 5/145; A61B 5/00; A61B 5/0402

(52) U.S. Cl. CPC A61B 5/0205; A61B 5/14551; A61B 5/14546; A61B 5/0245; A61B 5/742; A61B 5/0402; A61B 2560/04; A61B 5/7235

(57) ABSTRACT

A physiological signal measurement device includes a shell, a pair of induction sheets mounted to the shell, and a circuit board assembly mounted in the shell. The circuit board assembly includes a microprocessor, a photoplethysmography sensor electrically connected with the microprocessor, and an electrocardio signal sensor. The photoplethysmography sensor senses photoplethysmography signals of blood vessels reflected by the finger parts. The electrocardio signal sensor is electrically connected with the microprocessor and the pair of the induction sheets. The pair of the induction sheets respectively contact with finger parts of two hands to form a loop for sensing trace amounts of electrical signals generated from heart beats.

### CROSS-REFERENCE TO RELATED APPLICATION

The present application is based on, and claims priority form, Taiwan Patent Application No. 106101593, filed Jan. 17, 2017, the disclosure of which is hereby incorporated by reference herein in its entirety.

### BACKGROUND OF THE INVENTION

### 1. Field of the Invention

The present invention generally relates to a device and an algorithm applied therein, and more particularly to a physiological signal measurement device and a blood oxygen concentration algorithm applied therein.

### 2. The Related Art

With the development of information technologies, a physiological signal measurement device has been used more and more widely. Physiological signals of a user are measured by virtue of the physiological signal measurement device. The physiological signals include blood pressure signals, blood oxygen signals and electrocardio signals for monitoring health conditions of the user in real time.

However, a volume of the physiological signal measurement device is usually larger that makes the physiological signal measurement device need to be used at home. As a result, the physiological signals of the user doing outdoor sports are inconveniently measured in the real time.

Thus, how to design an innovative physiological signal measurement device has become a problem which need be solved by an inventor, the innovative physiological signal measurement device is carried conveniently, and is capable of measuring the physiological signals in the real time.

### SUMMARY OF THE INVENTION

An object of the present invention is to provide a physiological signal measurement device contacting with finger parts of two hands. The physiological signal measurement device includes a shell, a pair of induction sheets mounted to the shell, and a circuit board assembly mounted in the shell. The circuit board assembly includes a microprocessor, a photoplethysmography sensor electrically connected with the microprocessor, and an electrocardio signal sensor. The photoplethysmography sensor senses photoplethysmography signals of blood vessels reflected by the finger parts. The electrocardio signal sensor is electrically connected with the microprocessor and the pair of the induction sheets. The pair of the induction sheets respectively contact with the finger parts of the two hands to form a loop for sensing trace amounts of electrical signals generated from heart beats.

Another object of the present invention is to provide a blood oxygen concentration algorithm applied in a physiological signal measurement device. The physiological signal measurement device includes a photoplethysmography sensor. The photoplethysmography sensor includes red light and infrared light. Specific steps of the blood oxygen concentration algorithm are described hereinafter. An optical signal pulsation waveform is generated by virtue of oxyhemoglobins and hemoglobins of blood affecting light absorbance. The red light and the infrared light have different absorbance coefficients in the oxyhemoglobins and the hemoglobins to generate different AC signals with pulsation changes and DC signals with slow changes. AC signals denote alternating component signals, and DC signals denote direct component signals. Do a regression analysis with a R value by virtue of recording a lot of samples to obtain a linear coefficient of R corresponding to a blood oxygen concentration in accordance with Beer-Lambert Law. The R value is obtained by virtue of a formula expressed as: “R=(AC of RED/DC of RED)/(AC of IR/DC of IR)”. AC of RED denotes alternating component amplitude of the red light. DC of RED denotes direct component amplitude of the red light. AC of IR denotes alternating component amplitude of the infrared light. And DC of IR denotes direct component amplitude of the infrared light. SBP=a1×PWV+b1×BMI+c1, PWV=Height/(2×PTT). PWV denotes a pulse wave velocity. SBP denotes systolic blood pressure. PTT denotes pulse transmit time, and BMI denotes a body mass index. Calculate constants of a1 and b1 by means of obtaining SBP values, PTT values, Height values and BMI values of a mass of different users and applying a predicted model of monadic linear regression analysis method, so that the blood oxygen concentration is capable of being calculated by virtue of a formula expressed as: (% SPO2)=a1×R+b1. SPO2 denotes pulse oxygen saturation.

As described above, the physiological signal measurement device completes measuring physiological signals which include heart rate signals, blood pressure signals, blood oxygen concentration signals and so on of the users in the real time by virtue of the photoplethysmography sensor and the electrocardio signal sensor of the circuit board assembly. Furthermore, the shell of the physiological signal measurement device is of the card shape, so a volume of the physiological signal measurement device is smaller for being carried conveniently to be used outside and at home. As a result, the physiological signals of the user doing outdoor sports is conveniently measured in the real time.

### BRIEF DESCRIPTION OF THE DRAWINGS

The present invention will be apparent to those skilled in the art by reading the following description, with reference to the attached drawings, in which:

FIG. 1 is a perspective view of a physiological signal measurement device in accordance with a preferred embodiment of the present invention;

FIG. 2 is an exploded perspective view of the physiological signal measurement device of FIG. 1;

FIG. 3 is a perspective view of an upper shell of the physiological signal measurement device of FIG. 2;

FIG. 4 is a block diagram of a circuit board assembly of the physiological signal measurement device of FIG. 2; and

FIG. 5 is a flow chart of a blood oxygen concentration algorithm applied in the physiological signal measurement device in accordance with the preferred embodiment of the present invention.

### DETAILED DESCRIPTION OF THE PREFERRED EMBODIMENT

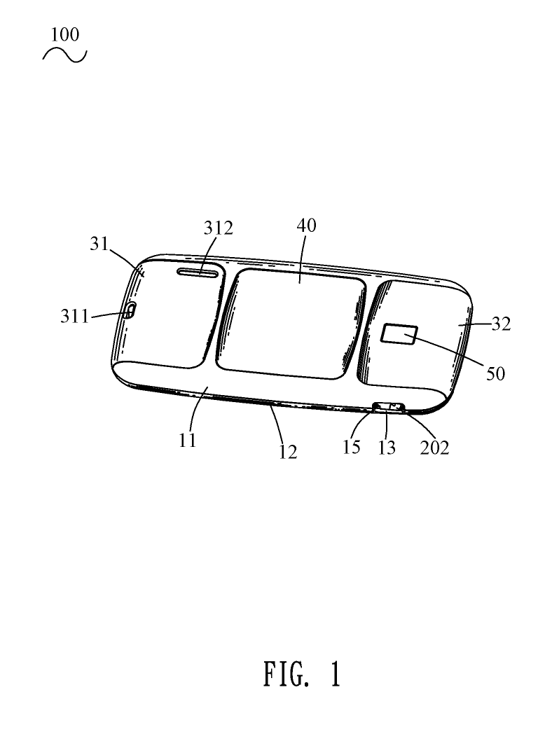

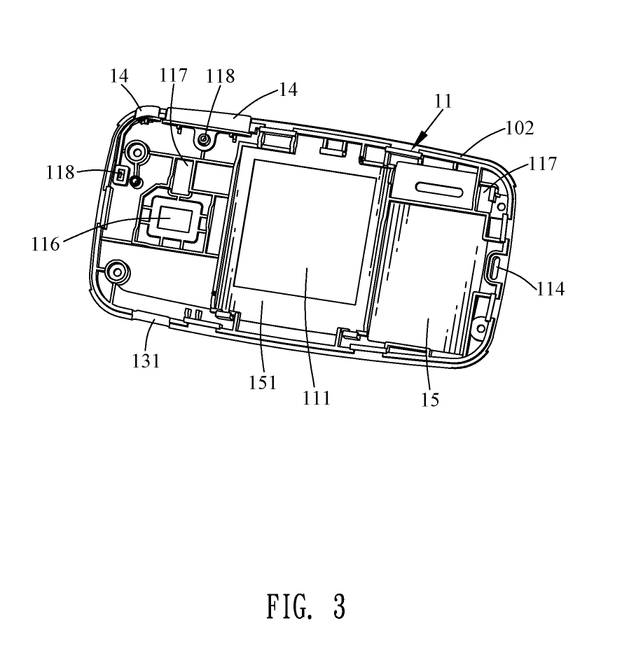

With reference to FIG. 1 to FIG. 3, a physiological signal measurement device 100 in accordance with a preferred embodiment of the present invention is shown. A blood oxygen concentration algorithm is applied in the physiological signal measurement device 100. The physiological signal measurement device 100 includes a shell 10, a circuit board assembly 20, a pair of induction sheets 30, a screen cover 40 and an optical sensor cover 50.

With reference to FIG. 1 to FIG. 3, the shell 10 is of a card shape. The shell 10 includes an upper shell 11 and a lower shell 12. The upper shell 11 has a top surface 101, and a bottom surface 102 opposite to the top surface 101. A lower portion of the upper shell 11 opens an upper receiving space 151 penetrating through a middle of the bottom surface 102 of the upper shell 11 in a downward direction. Two opposite ends of the top surface 101 of the upper shell 11 are recessed in the downward direction to form a first recess 112 and a second recess 113. A bottom wall of the first recess 112 of the upper shell 11 opens an upper sling hole 114 and an internal loudspeaker hole 115. A bottom wall of the second recess 113 defines a locating groove 119. A bottom wall of the locating groove 119 opens an optical sensor hole 116 communicated with the locating groove 119. In this preferred embodiment, a middle of the bottom wall of the locating groove 119 opens the optical sensor hole 116.

The bottom wall of the first recess 112 and the bottom wall of the second recess 113 open two perforations 117, respectively. Several portions of a bottom of the upper shell 11 protrude in the downward direction to form a plurality of protruding pillars 118. Several portions of a bottom surface of a top wall of the upper receiving space 151 protrude in the downward direction to form the plurality of protruding pillars 118. In this preferred embodiment, the upper sling hole 114, the internal loudspeaker hole 115, the optical sensor hole 116 and the two perforations 117 are communicated with the upper receiving space 151. The upper shell 11 is covered on the lower shell 12 to form a receiving space 15 between the lower shell 12 and the upper shell 11. The lower shell 12 has a superface 103 facing the bottom surface 102 of the upper shell 11. An upper portion of the lower shell 12 opens a lower receiving space 152 penetrating through a middle of the superface 103 of the lower shell 12 in an upward direction.

The lower receiving space 152 is corresponding to and communicated with the upper receiving space 151 to form the receiving space 15. An upper portion of the upper shell 11 opens an opening 111 penetrating through a middle of the top surface 101 of the upper shell 11 in the upward direction and extending to the upper receiving space 151 of the receiving space 15 in the downward direction. The opening 111 is communicated with the upper receiving space 151. The opening 111 is located between the first recess 112 and the second recess 113. The lower shell 12 opens a lower sling hole 121 corresponding to the upper sling hole 114 of the upper shell 11. A periphery of the bottom surface 102 of the upper shell 11 is connected with a periphery of the superface 103 of the lower shell 12. The downward direction is opposite to the upward direction.

A periphery of the shell 10 opens a plurality of assembling grooves 13. Several portions of the periphery of the bottom surface 102 of the upper shell 11 and several portions of the periphery of the superface 103 of the lower shell 12 are recessed in opposite directions to form a plurality of upper assembling grooves 131 and a plurality of lower assembling grooves 132. In this preferred embodiment, several portions of two opposite sides of the periphery of the bottom surface 102 of the upper shell 11 and several portions of two opposite sides of the periphery of the superface 103 of the lower shell 12 are recessed in the opposite directions to form the plurality of the upper assembling grooves 131 and the plurality of the lower assembling grooves 132, respectively. The plurality of the lower assembling grooves 132 are matched with the corresponding plurality of the upper assembling grooves 131 to form the plurality of the assembling grooves 13.

The shell 10 further includes a plurality of buttons 14. Each of the plurality of the buttons 14 is assembled in one of the plurality of the assembling grooves 13. Each of the plurality of the buttons 14 includes a pressing portion 141, a ring-shaped assembling portion 142, and a connecting portion 143 connected between the pressing portion 141 and the assembling portion 142. The assembling portion 142 of each of the plurality of the buttons 14 is assembled to one of the protruding pillars 118 of the upper shell 11. The connecting portion 143 of each of the plurality of the buttons 14 is received in the upper receiving space 151 of the upper shell 11. The pressing portion 141 of each of the plurality of the buttons 14 is assembled to the one of the plurality of the assembling grooves 13 of the shell 10.

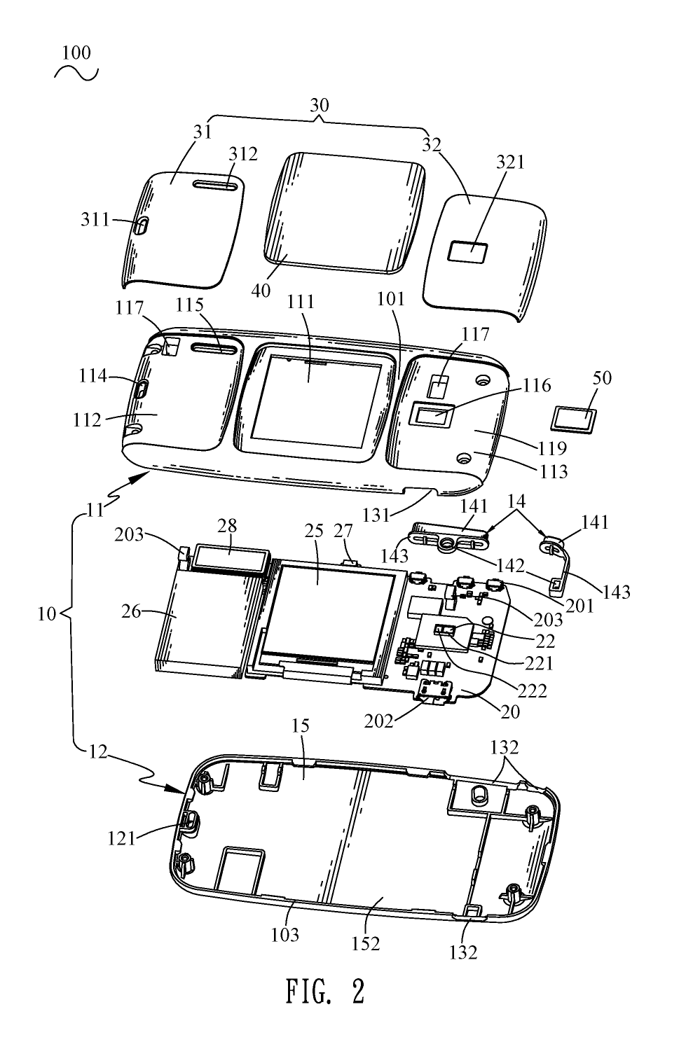

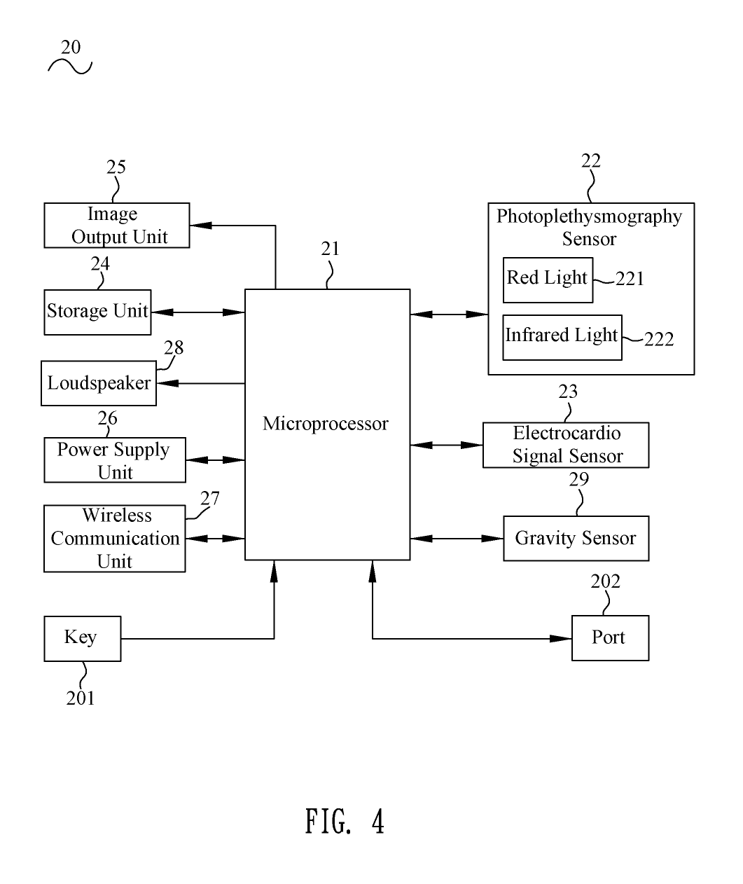

Referring to FIG. 1, FIG. 2 and FIG. 4, the circuit board assembly 20 is mounted in the shell 10. The circuit board assembly 20 is received in the receiving space 15. The circuit board assembly 20 includes a microprocessor 21, a photoplethysmography sensor 22, an electrocardio signal sensor 23, a storage unit 24, an image output unit 25, a power supply unit 26, a wireless communication unit 27, a loudspeaker 28 and a gravity sensor 29.

The photoplethysmography sensor 22 is electrically connected with the microprocessor 21. In use, the physiological signal measurement device 100 contacts with finger parts of two hands of a user. The photoplethysmography sensor 22 senses photoplethysmography signals of blood vessels reflected by the finger parts, and blood pressure values and blood oxygen concentration values are calculated by the microprocessor 21. In this preferred embodiment, the photoplethysmography sensor 22 is assembled on a top of the circuit board assembly 20 and is located at one side of the second recess 113. Specifically, the photoplethysmography sensor 22 is assembled in the optical sensor hole 116 of the second recess 113 of the upper shell 11 and exposed in the locating groove 119. The optical sensor cover 50 is assembled in the locating groove 119 and is covered on the photoplethysmography sensor 22. In use, the photoplethysmography signals of the finger parts are sensed by the photoplethysmography sensor 22 and are transmitted to the microprocessor 21 for a calculation.

The pair of the induction sheets 30 mounted to the shell 10. The electrocardio signal sensor 23 is electrically connected with the microprocessor 21 and the pair of the induction sheets 30. In use, the pair of the induction sheets 30 respectively contact with the finger parts of the two hands of the user to form a loop for sensing trace amounts of electrical signals generated from heart beats, and heart rate values are calculated by the microprocessor 21. Specifically, the pair of the induction sheets 30 are respectively pressed by thumbs of the two hands of the user, at the moment, the physiological signal measurement device 100, the two hands and a body of the user form a measurement loop. The electrocardio signal sensor 23 senses the trace amounts of the electrical signals generated from the heart beats by virtue of the pair of the induction sheets 30 contacting with the finger parts of the two hands, and the trace amounts of the electrical signals are transmitted to the microprocessor 21 for being calculated.

In this preferred embodiment, specific steps of a blood pressure calculation method applied in the physiological signal measurement device 100 are described as follows. Set the photoplethysmography sensor 22 and the electrocardio signal sensor 23, and establish a calculation formula of SBP (Systolic Blood Pressure) value which is expressed as: “SBP=a1×PWV+b1×BMI+c1”, and a calculation formula of DBP (Diastolic Blood Pressure) value which is expressed as: “DBP=d1×SBP+e1”. PWV=Height/(2×PTT), PWV denotes a pulse wave velocity, SBP denotes systolic blood pressure, DBP denotes diastolic blood pressure, PTT denotes pulse transmit time, and BMI denotes a body mass index. Calculate constants of a1, b1, c1, d1 and e1 by means of obtaining SBP values, DBP values, PTT values, Height values and BMI values of a mass of different users and applying a predicted model of monadic linear regression analysis method, the calculation formulas: “SBP=a1×PWV+b1×BMI+c1” and “DBP=d1×SBP+e1” are written to the microprocessor 21.

In use, sample a photoplethysmography pulse signal and an electrocardio signal of the user, and calculate the PTT value of the user. Input the Height value and the BMI value of the user into the physiological signal measurement device 100. The microprocessor 21 is capable of calculating the SBP value of the user, and then the DBP value of the user is directly calculated by virtue of the SBP value being applied in the calculation formula of the DBP value.

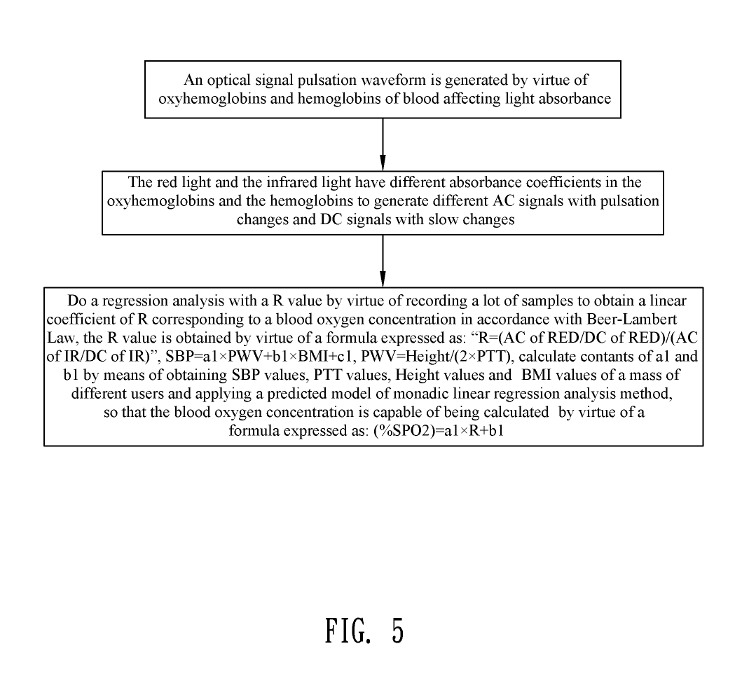

Referring to FIG. 1 to FIG. 5, in this preferred embodiment, the photoplethysmography sensor 22 includes red light 221 and infrared light 222. Specific steps of the blood oxygen concentration algorithm applied in the physiological signal measurement device 100 are described as follows.

Firstly, an optical signal pulsation waveform is generated by virtue of oxyhemoglobins (HbO2) and hemoglobins (Hb) of blood affecting light absorbance.

Secondly, the red light 221 and the infrared light 222 have different absorbance coefficients in the oxyhemoglobins and the hemoglobins to generate different AC signals with pulsation changes and DC signals with slow changes, AC signals denote alternating component signals, and DC signals denote direct component signals.

Thirdly, do a regression analysis with a R value by virtue of recording a lot of samples to obtain a linear coefficient of R corresponding to a blood oxygen concentration in accordance with Beer-Lambert Law, the R value is obtained by virtue of a formula expressed as: “R=(AC of RED/DC of RED)/(AC of IR/DC of IR)”, AC of RED denotes alternating component amplitude of the red light 221, DC of RED denotes direct component amplitude of the red light 221, AC of IR denotes alternating component amplitude of the infrared light 222, and DC of IR denotes direct component amplitude of the infrared light 222, so that the blood oxygen concentration is capable of being calculated by virtue of a formula expressed as: (% SPO2)=a1×R+b1, SPO2 denotes pulse oxygen saturation.

The storage unit 24 is electrically connected with the microprocessor 21 for storing measured data of the physiological signal measurement device 100 which include data calculated by the microprocessor 21 in the storage unit 24.

The image output unit 25 is electrically connected with the microprocessor 21 for displaying the measured data of the physiological signal measurement device 100 which include the data calculated by the microprocessor 21 in real time. In this preferred embodiment, the image output unit 25 is disposed to the top of the circuit board assembly 20 and is fixed in the opening 111 of the upper shell 11. The screen cover 40 is assembled in the opening 111 and is covered on the image output unit 25.

The power supply unit 26 is electrically connected with the microprocessor 21 to provide power signals for the circuit board assembly 20 to make the circuit board assembly 20 work.

The wireless communication unit 27 is electrically connected with the microprocessor 21 for making the measured data of the physiological signal measurement device 100 transmitted to a peripheral equipment in the real time.

The loudspeaker 28 is electrically connected with the microprocessor 21 for making the data calculated by the microprocessor 21 transmitted outside by sound signals. In this preferred embodiment, the loudspeaker 28 is disposed to the top of the circuit board assembly 20, and is located under the first recess 112 of the upper shell 11. Specifically, the loudspeaker 28 is mounted under the internal loudspeaker hole 115 of the first recess 112 of the upper shell 11.

The gravity sensor 29 is electrically connected with the microprocessor 21. Signals sensed by the gravity sensor 29 are provided for the microprocessor 21 to calculate data of step calculations and so on.

The circuit board assembly 20 further includes a plurality of keys 201, a port 202 and two conductive elements 203. The plurality of the keys 201 and the port 202 are disposed to a peripheral edge of the circuit board assembly 20. The two conductive elements 203 are disposed to two opposite ends of the top of the circuit board assembly 20. In this preferred embodiment, the plurality of the keys 201 and the port 202 are disposed to two opposite sides of the peripheral edge of the circuit board assembly 20. The pressing portions 141 of the plurality of the buttons 14 of the shell 10 are disposed on the plurality of the keys 201. Each of the plurality of the keys 201 has functions of turning on or switching off, adjusting volumes, going forward and receding, and so on. The port 202 is disposed to and corresponding to one of the plurality of the assembling grooves 13 of the shell 10. The plurality of the keys 201 are disposed in the other assembling grooves 13 of the shell 10. The two conductive elements 203 are received in and project out of the two perforations 117, respectively. The physiological signal measurement device 100 proceeds charging and data transmissions by virtue of the port 202.

The pair of the induction sheets 30 include a first pole piece 31 and a second pole piece 32. The first pole piece 31 opens an external sling hole 311 corresponding to the upper sling hole 114 of the first recess 112 of the upper shell 11. The first pole piece 31 opens an external loudspeaker hole 312 corresponding to the internal loudspeaker hole 115 of the first recess 112 of the upper shell 11. The second pole piece 32 opens an external sensor hole 321 corresponding to and communicated with the optical sensor hole 116 of the second recess 113 of the upper shell 11. The first pole piece 31 is assembled in the first recess 112 of the upper shell 11 and is electrically connected with one of the two conductive elements 203 of the circuit board assembly 20 by virtue of one of the two perforations 117. The external sling hole 311 of the first pole piece 31 is corresponding to and communicated with the upper sling hole 114 of the upper shell 11. The external loudspeaker hole 312 of the first pole piece 31 is corresponding to and communicated with the internal loudspeaker hole 115 of the upper shell 11. The second pole piece 32 is assembled in the second recess 113 and is electrically connected with the other conductive element 203 of the circuit board assembly 20 by virtue of the other perforation 117. The optical sensor cover 50 is exposed in the external sensor hole 321.

As described above, the physiological signal measurement device 100 completes measuring physiological signals which include heart rate signals, blood pressure signals, blood oxygen concentration signals and so on of the users in the real time by virtue of the photoplethysmography sensor 22 and the electrocardio signal sensor 23 of the circuit board assembly 20. Furthermore, the shell 10 of the physiological signal measurement device 100 is of the card shape, so a volume of the physiological signal measurement device 100 is smaller for being carried conveniently to be used outside and at home. As a result, the physiological signals of the user doing outdoor sports is conveniently measured in the real time.

What is claimed is:

1. A physiological signal measurement device contacting with finger parts of two hands, comprising:
a shell;
a pair of induction sheets mounted to the shell; and
a circuit board assembly mounted in the shell, including:
a microprocessor,
a photoplethysmography sensor electrically connected with the microprocessor, the photoplethysmography sensor sensing photoplethysmography signals of blood vessels reflected by the finger parts; and
an electrocardio signal sensor electrically connected with the microprocessor and the pair of the induction sheets, the pair of the induction sheets respectively contacting with the finger parts of the two hands to form a loop for sensing trace amounts of electrical signals generated from heart beats.

2. The physiological signal measurement device as claimed in claim 1, wherein the circuit board assembly further includes an image output unit electrically connected with the microprocessor for displaying measured data of the physiological signal measurement device in real time.

3. The physiological signal measurement device as claimed in claim 1, wherein the circuit board assembly further includes a power supply unit electrically connected with the microprocessor to provide power signals for the circuit board assembly to make the circuit board assembly work.

4. The physiological signal measurement device as claimed in claim 1, wherein the circuit board assembly further includes a wireless communication unit electrically connected with the microprocessor for making measured data of the physiological signal measurement device transmitted to a peripheral equipment in real time.

5. The physiological signal measurement device as claimed in claim 1, wherein the circuit board assembly further includes a storage unit electrically connected with the microprocessor for storing measured data of the physiological signal measurement device in the storage unit.

6. The physiological signal measurement device as claimed in claim 1, wherein the circuit board assembly further includes a gravity sensor electrically connected with the microprocessor, signals sensed by the gravity sensor are provided for the microprocessor to calculate.

7. The physiological signal measurement device as claimed in claim 1, wherein the circuit board assembly further includes a loudspeaker electrically connected with the microprocessor for making data calculated by the microprocessor transmitted outside by sound signals.

8. The physiological signal measurement device as claimed in claim 1, wherein the shell includes an upper shell and a lower shell, the upper shell is covered on the lower shell to form a receiving space between the lower shell and the upper shell, the circuit board assembly is received in the receiving space.

9. The physiological signal measurement device as claimed in claim 8, wherein the shell is of a card shape, a lower portion of the upper shell opens an upper receiving space penetrating through a middle of a bottom surface of the upper shell in a downward direction, an upper portion of the lower shell opens a lower receiving space penetrating through a middle of a superface of the lower shell in an upward direction opposite to the downward direction, the lower receiving space is corresponding to and communicated with the upper receiving space to form the receiving space.

10. The physiological signal measurement device as claimed in claim 8, wherein two opposite ends of a top surface of the upper shell are recessed in a downward direction to form a first recess and a second recess, the pair of the induction sheets include a first pole piece and a second pole piece, the first pole piece is assembled in the first recess, the second pole piece is assembled in the second recess.

11. The physiological signal measurement device as claimed in claim 10, wherein a bottom wall of the first recess of the upper shell opens an upper sling hole, the lower shell opens a lower sling hole corresponding to the upper sling hole of the upper shell.

12. The physiological signal measurement device as claimed in claim 10, wherein a bottom wall of the first recess and a bottom wall of the second recess open two perforations, respectively, the circuit board assembly further includes two conductive elements disposed to two opposite ends of a top of the circuit board assembly, the two conductive elements are received in and project out of the two perforations, respectively, the first pole piece is electrically connected with one of the two conductive elements by virtue of one of the two perforations, the second pole piece is electrically connected with the other conductive element by virtue of the other perforation.

13. The physiological signal measurement device as claimed in claim 12, further comprising an optical sensor cover, a bottom wall of the second recess defining a locating groove, a bottom wall of the locating groove opening an optical sensor hole communicated with the locating groove, the circuit board assembly further including a photoplethysmography sensor assembled on the top of the circuit board assembly and located at one side of the second recess, the photoplethysmography sensor being assembled in the optical sensor hole and exposed in the locating groove, the optical sensor cover being assembled in the locating groove and being covered on the photoplethysmography sensor, the second pole piece opening an external sensor hole corresponding to and communicated with the optical sensor hole, the optical sensor cover being exposed in the external sensor hole.

14. The physiological signal measurement device as claimed in claim 8, further comprising a screen cover, an upper portion of the upper shell opening an opening penetrating through a middle of a top surface of the upper shell in an upward direction and extending to the receiving space in a downward direction, the circuit board assembly further including an image output unit, the image output unit being disposed to a top of the circuit board assembly and being fixed in the opening, the screen cover being assembled in the opening and being covered on the image output unit.

15. The physiological signal measurement device as claimed in claim 8, wherein a periphery of the shell opens a plurality of assembling grooves, the shell further includes a plurality of buttons, each of the plurality of the buttons is assembled in one of the plurality of the assembling grooves.

16. The physiological signal measurement device as claimed in claim 15, wherein several portions of a periphery of a bottom surface of the upper shell and several portions of a periphery of a superface of the lower shell are recessed in opposite directions to form a plurality of upper assembling grooves and a plurality of lower assembling grooves, the plurality of the lower assembling grooves are matched with the corresponding pluality of the upper assembling grooves to form the plurality of the assembling grooves, each of the buttons includes a pressing portion, the pressing portion of each of the plurality of the buttons is assembled to the one of the plurality of the assembling grooves.

17. The physiological signal measurement device as claimed in claim 16, wherein the circuit board assembly further includes a plurality of keys, the pressing portions of the plurality of the buttons of the shell are disposed on the plurality of the keys.

18. The physiological signal measurement device as claimed in claim 15, wherein several portions of a bottom of the upper shell protrude in a downward direction to form a plurality of protruding pillars, each of the plurality of the buttons includes a ring-shaped assembling portion, the assembling portion of each of the plurality of the buttons is assembled to one of the protruding pillars of the upper shell.

19. The physiological signal measurement device as claimed in claim 15, wherein the circuit board assembly further includes a port disposed to a peripheral edge of the circuit board assembly, the port is disposed to and corresponding to one of the plurality of the assembling grooves of the shell.

20. A blood oxygen concentration algorithm applied in a physiological signal measurement device, the physiological signal measurement device including a photoplethysmography sensor, the photoplethysmography sensor including red light and infrared light, the blood oxygen concentration algorithm comprising the steps of:
an optical signal pulsation waveform being generated by virtue of oxyhemoglobins and hemoglobins of blood affecting light absorbance;
the red light and the infrared light having different absorbance coefficients in the oxyhemoglobins and the hemoglobins to generate different AC signals with pulsation changes and DC signals with slow changes, AC signals denoting alternating component signals, and DC signals denoting direct component signals; and
doing a regression analysis with a R value by virtue of recording a lot of samples to obtain a linear coefficient of R corresponding to a blood oxygen concentration in accordance with Beer-Lambert Law, the R value being obtained by virtue of a formula expressed as: “R=(AC of RED/DC of RED)/(AC of IR/DC of IR)”, AC of RED denoting alternating component amplitude of the red light, DC of RED denoting direct component amplitude of the red light, AC of IR denoting alternating component amplitude of the infrared light, and DC of IR denoting direct component amplitude of the infrared light, SBP=a1×PWV+b1×BMI+c1, PWV=Height/(2×PTT), PWV denoting a pulse wave velocity, SBP denoting systolic blood pressure, PTT denoting pulse transmit time, and BMI denoting a body mass index, calculating constants of a1 and b1 by means of obtaining SBP values, PTT values, Height values and BMI values of a mass of different users and applying a predicted model of monadic linear regression analysis method, so that the blood oxygen concentration is capable of being calculated by virtue of a formula expressed as: (% SPO2)=a1×R+b1, SPO2 denoting pulse oxygen saturation.
